(19) United States
(12) Patent Application Publication
(10) Pub. No.: US 2023/0240802 A1
(43) Pub. Date: Aug. 3, 2023

(54) DEVICES AND METHODS OF TREATING SLEEP AND AWAKE BRUXISM

(71) Applicant: PerioTech, LLC, Palm Beach Gardens, FL (US)

(72) Inventors: Richard JOHNSON, Palm Beach Gardens, FL (US); Nicholas PURO, Sunny Isles Beach, FL (US)

(21) Appl. No.: 17/587,686

(22) Filed: Jan. 28, 2022

(51) Int. Cl. A61C 7/00; A61C 7/08; A61F 5/56

(52) U.S. Cl. CPC A61C 7/008; A61C 7/08; A61F 5/566; A61F 2005/563

(57) ABSTRACT

A device and method for treating awake and sleep bruxism in a patient includes a vibrational dental device having a mouthpiece for contacting the dentition. Instructions are provided for waking use of the vibrational dental device by placing the mouthpiece over the dentition, applying a vibratory force during a predetermined number of sessions throughout a predetermined treatment period, wherein the incidence of awake and sleep bruxism is decreased compared to without vibratory treatment.

### TECHNICAL FIELDS

This disclosure concerns treatments of musculoskeletal tension conditions, more specifically, devices and methods to facilitate treatment of sleep and/or awake bruxism using intraoral mechanical vibration.

### BACKGROUND

Bruxism is repetitive clenching or grinding of teeth by action of the jaw muscle. While it is generally thought that most people brux during sleep, repetitive and forceful bruxing can lead to several negative health effects. Certain medications can cause bruxism, such as the antidepressants fluoxetine and paroxetine. It is unclear what causes non-iatrogenic sleep bruxism, but high stress seems to be correlated. Certain personality types have also been identified as potentially more susceptible to sleep bruxism.

Awake bruxism and clenching have been linked to stress levels. Although a positive relationship has been found between awake bruxism and levels of anxiety, this has not been found between sleep bruxism and anxiety. Over time, clenching or bruxing becomes a habit that compromises the orofacial region, with an association to dental pain, facial musculature pain as well as strain on the temporomandibular joint (TMJ).

Sleep bruxism can lead to damage of the dentition, such as abraded, chipped, cracked or sensitive teeth, facial pain and tension including headaches, and jaw maladies including dislocation and locking of the temporomandibular joint (TMJ) or clicking or popping of the jaw. Severe abrasion of the teeth can involve degradation of the tooth enamel, exposing the underlying dentin or the formation of flat spots, or facets, on the occlusal surfaces of the teeth. Soft oral tissues can also be affected, such as through tongue indentations and damage to buccal tissues.

There is a difference in bruxism when asleep and awake. Sleep bruxism is a masticatory muscle activity when sleeping, characterized as either rhythmic (phasic) or non-rhythmic (tonic). Whereas, awake bruxism is a masticatory muscle activity when awake characterized by repetitive or sustained tooth contact that may also involve bracing or thrusting of the mandible. Awake bruxism may be due to emotions such as anxiety, stress, anger, frustration or tension, or it may be a coping strategy or a habit during deep concentration. Yet, many patients as mentioned suffer with awake clenching and bruxism related to day-time stress and utilization of an oral appliance for bruxism does not eliminate or lessen the neural muscle stimulation that adds to further clenching and bruxism. It has been reported that 20-38% of the population has periodic or daily awake bruxism.

Currently, there is no known cure for bruxism. The standard of care at the present time involves relaxation exercises and mouth-rest-training techniques, mouthguards worn at night, and biofeedback devices that provide a stimulus to a user in response to a sensed bruxing of the teeth which sleeping. These stimuli can be a sound, a light electrical shock or a vibration. Disadvantage of these types of devices include difficulty for some patients getting accustomed to sleeping with an alert apparatus in their mouths and decreasing effectiveness of the alert in stopping bruxism as the patient becomes desensitized to the corrective stimulus.

### SUMMARY

According to an exemplary embodiment of the present disclosure, a method for treating bruxism is described. The method includes identifying a patient having bruxism, providing to the patient a vibrational dental device having a mouthpiece for contacting the dentition, and providing instructions for using the vibrational dental device. The instruction includes placing the mouthpiece over the dentition and applying a vibratory force for a period of time. Low-magnitude high-frequency vibration (LMHFV) applied to the dentition travels through the mandible and craniofacial bones and converges at the TMJ. The pterygoid and masseter muscles are subject to vibration, aiding in their relaxation. The inferior muscles of the TMJ are also vibrated, including the digastric, mylohyoid and infrahyoid, aiding in their relaxation.

According to yet another exemplary embodiment of the present disclosure, a method for preventing bruxism is described. The method includes identifying a patient taking fluoxetine, paroxetine or related drugs associated with iatrogenic bruxism, providing to the patient a vibrational dental device having a mouthpiece for contacting the dentition and/or the dental implant, and providing instructions for using the vibrational dental device. The instruction includes placing the mouthpiece over the dentition and applying a vibratory force during a predetermined number of sessions throughout a predetermined treatment period.

Additional features and advantages of the disclosed embodiments will be set forth in part in the description that follows, and in part will be obvious from the description, or may be learned by practice of the disclosed embodiments. The features and advantages of the disclosed embodiments will be realized and attained by the elements and combinations particularly pointed out in the appended claims.

It is to be understood that both the foregoing general description and the following detailed description are examples and explanatory only and are not restrictive of the disclosed embodiments as claimed.

### BRIEF DESCRIPTION OF THE DRAWINGS

The accompanying drawings constitute a part of this specification. The drawings illustrate several embodiments of the present disclosure and, together with the description, serve to explain the principles of the disclosed embodiments as set forth in the accompanying claims.

The drawings are not necessarily to scale or exhaustive. Instead, emphasis is generally placed upon illustrating the principles of the inventions described herein. The accompanying drawings, which are incorporated in and constitute a part of this specification, illustrate several embodiments consistent with the disclosure and together with the description, serve to explain the principles of the disclosure. In the drawings:

FIG. 1A depicts an illustrative vibrational dental device according to one aspect of the disclosure;

FIG. 1B depicts an illustrative vibrational dental device, such as that depicted in FIG. 1A placed in the mouth of a user, according to one aspect of the disclosure;

FIG. 2 is a chart showing the effects over time on patient-reported bruxism while using LMHFV.

Reference will now be made in detail to exemplary embodiments. Unless otherwise defined, technical or scientific terms have the meaning commonly understood by one of ordinary skill in the art. The disclosed embodiments are described in sufficient detail to enable those skilled in the art to practice the disclosed embodiments. It is to be understood that other embodiments may be utilized and that changes may be made without departing from the scope of the disclosed embodiments. Thus, the materials, methods, and examples are illustrative only and are not intended to be necessarily limiting.

### DETAILED DESCRIPTION

It is to be understood that both the foregoing general description and the following detailed description are exemplary and explanatory only and are not restrictive of the claims.

The disclosed embodiments relate to devices, systems, and methods for treating sleep bruxism. Advantageously, embodiments of the present disclosure can be implemented to lower the incidence of sleep bruxism than without. This is surprising in light of the generally held view that the only non-medical means of treating sleep bruxism by behavioral modification. According to some aspects of the present disclosure, use of intraoral low-magnitude high-frequency vibration (LMHFV) while awake in a patient diagnosed with sleep bruxism can lower the incidences of sleep bruxism as compared to without treatment, lessening the risk of damage to teeth and lowering stress on the jaw and TMJ.

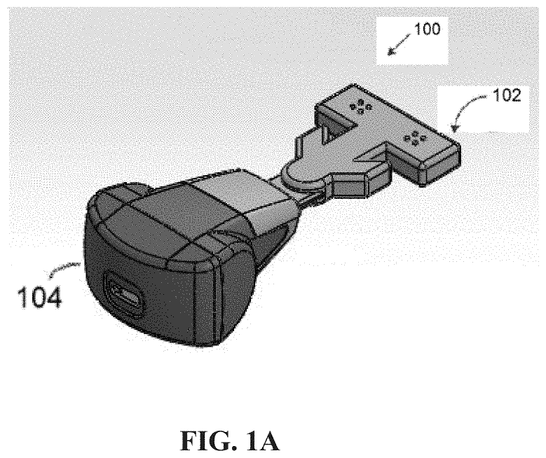

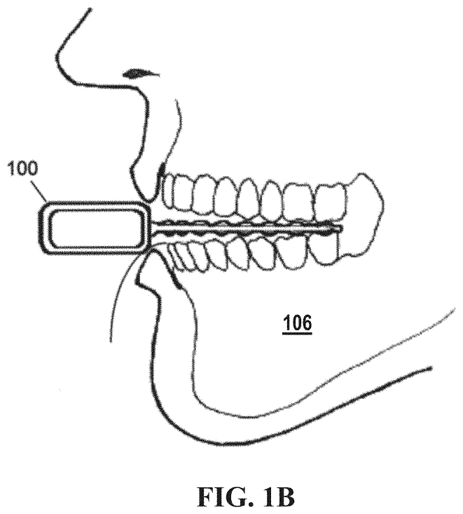

In an aspect, with reference to FIGS. 1A and 1B, use of the device 100 providing LMHFV for 5 minutes daily before bedtime can decrease the incidence of sleep bruxism. Without being bound by theory, it is speculated that vibration applied to the dentition of a patient with sleep bruxism will, through bone conduction of the vibration, help to relax and calm the craniofacial and mandibular muscles associated with the TMJ. Further advantageously, stress is relieved in the patient with sleep bruxism, contributing to implant stability and better overall oral health. Additionally advantageously, lower stress and relaxation may help decrease the incidence of related maladies such as facial pain, stress headaches and migraine.

LMHFV therapy according to the present disclosure is also advantageously configured to help alleviate bruxism in patients receiving medical treatments tending to increase the incidence of bruxism. In an aspect, patients receiving anti-depressants such as fluoxetine and paroxetine can be treated with LMHFV to help combat these drugs' correlation with increased bruxism.

Described herein are LMHFV dental devices, which in certain embodiments include a mouthpiece configured to transmit vibration to all or a portion of the patient's teeth. In some embodiments, the appliance can be configured to engage with a patient's teeth alone or can be configured to engage with a patient's teeth and gums. The mouthpiece may gently engage with the gum line at or below the gingival margin to provide stimulation to the soft tissue. Such gentle stimulation can help to increase blood flow and other cells of repair to the site, in addition to that provided by vibration conducted through neighboring teeth and tissue structures. The appliance can be configured to stimulate one or more teeth, the gum line only, or both one or more teeth and the gumline.

Referring to FIGS. 1A-1B, an exemplary dental device 100 includes a mouthpiece 102 operatively connected to a housing 104. The mouthpiece 102 can be separable from the housing 104 for interchangeability between users or for ease of cleaning. The mouthpiece 102 can include one or more oral tissue-contacting portion, such as a biteplate or probe for contacting teeth, gums or other oral tissues. As shown, in FIGS. 1A, the mouthpiece can include a biteplate which can be appropriately shaped to cover occlusal surfaces of some or all of a patient's dentition. Other shapes for the mouthpiece are possible. For example, the mouthpiece can be configured to abut the lingual and buccal lateral sides of the alveolar ridge either with or without occlusal contact or, when no teeth are present, contact with gums overlying the alveolar ridge. A vibration generator can be located in the mouthpiece 102 or the housing 104 to vibrate the mouthpiece 102. In some embodiments (not shown) the vibrators can be mounted to the biteplate, and in still further embodiments the entire device can be intraoral. The housing 104 can also include the electronics to run the motor the vibrator, collect usage and device operation data, collect data from sensors in the mouthpiece or base, and store data in memory. The housing 104 can include a data interface which can be wired or wireless to allow a data connection to other devices. The housing 104 can also include a power interface to allow charging of any onboard power sources, such as batteries or capacitor banks. The mouthpiece 102 can be electrically interconnected to the housing 104. FIG. 1B depicts an illustrative dental device 100, such as that described above with reference to FIG. 1A, inserted in the mouth of a human user 106 and engaging the occlusal surfaces of the molars. The mouthpiece of the dental device 100 can, as described above, be sized and shaped to contact any dental tissue, including some or all of the teeth, specific regions of the gums, or both.

As is known in the art, the vibration generator can include an electric motor connected to an eccentric weight, or can be a piezo generator, as well as other known expedients. Accordingly, when the mouthpiece 102 is placed in a patient's mouth and the dental device is 100 turned on, the vibration of the mouthpiece 102 will place vibratory force repetitively on the teeth and/or other oral tissues.

LMHFV in one aspect helps alleviate ischemia and orofacial pain in the masseter and other muscles associated with the jaw. The use of LMHFV in the morning after bruxing can advantageously help relax the muscles involved in clenching and avoid soreness. The relaxation of the masticatory muscles as well as the clearing of post-exertion compounds from the muscles, such as lactose dehydrogenase, can decrease orofacial pain.

In some embodiments, the sleep bruxing patient can be instructed to use the appliance for a prescribed time and duration to decrease bruxism. In an example, the patient can be instructed to use the appliance for five minutes daily over a four-month period. The vibration can be applied along multiple axes or selected to be primarily on a single axis. The primary anatomic reference directions with reference to a standing human are superior-inferior (up and down), anterior-posterior (front to back), medial-lateral (side to side). Because mastication places loading on oral structures primarily in the superior-inferior direction through mandibular action, it may be advantageous to choose vibrational loading along other axes either separately or in combination.

Vibrational Bruxism Treatment Devices

According to an aspect of the present disclosure, a vibrational sleep bruxism treatment device that vibrates at one or more predetermined frequencies is provided. In some embodiments the vibrational frequency is fixed within a lower bound and an upper bound. The lower bound can be greater than about 110 Hz, 105 Hz, 100 Hz, 95 Hz, 90 Hz, 85 Hz, 80 Hz, 75 Hz, 70 Hz, 65 Hz, 60 Hz, 55 Hz, 50 Hz, 45 Hz, or less. The upper bound can be greater than about 115 Hz, 120 Hz, 125 Hz, 130 Hz, 135 Hz, 140 Hz, 145 Hz, 150 Hz, or more. In some embodiments, the frequency varies within a lower and an upper bound. In some embodiments two or more frequencies, fixed or varying, are employed.

In some embodiments the duration of a treatment session can be specified to be greater than about 30 seconds, 1 min, 2 min, 3 min, 4 min, 5 min, 6 min, 7 min, 8 min, 9 min, 10 min, 11 min, 12 min, 13 min, 14 min, 15 min, 16 min, 17 min, 18 min, 19 min, 20 min, or more; or specified to be less than about 20 min, 19 min, 18 min, 17 min, 16 min, 15 min, 14 min, 13 min, 12 min, 10 min, 9 min, 8 min, 7 min, 6 min, 5 min, 4 min, 3 min, 2 min, 1 min, 30 seconds, or less.

FIG. 1A depicts a vibrational bruxism device according to an example. The vibrational bruxism device can include a mouthpiece and a vibrational source connected to each other. The mouthpiece is configured to be provided between the occlusal surfaces of a user's teeth, and to be bite down by the user to contact the user's dentition during the treatment. The mouthpiece can cover at least the teeth or implant around which accelerating graft conversion is desired. The vibrational source is configured to provide vibration to the mouthpiece at a preset frequency and acceleration.

To achieve the maximum desired results of relaxation of the masticatory muscles, further studies are still needed to optimize the parameters of LMHFV. Such parameters may include frequency, acceleration, and dosage. Dosage may include duration per use, number of uses per day, or number of days of use, either consecutively or at a certain schedule.

In some embodiments, the vibrational source may be connected to the mouthpiece in such way that the vibration provided is in the sagittal plane of a user's mouth. A motor may be included in the vibrational source to provide such vibration. The motor may be of any suitable type known in the art. The motor, when in use, may be configured to provide vibration at a frequency as disclosed herein. The motor, when in use, may be further configured to provide vibration at an acceleration magnitude. In some embodiments the mouthpiece of a bruxism vibration device can have an acceleration within a lower bound and an upper bound. The lower bound can be greater than about 0.010 G, 0.015 G, 0.020 G, 0.025 G, 0.030 G, 0.035 G, 0.040 G, 0.045 G, 0.050 G, 0.055 G, 0.060 G, or more; or less than about 0.060 G, 0.055 G, 0.050 G, 0.045 G, 0.040 G, 0.035 G, 0.030 G, 0.025 G, 0.020 G, 0.015 G, 0.010 G, or less. The upper bound can be greater than about 0.07 G, 0.08 G, 0.09 G, 0.10 G, 0.11 G, 0.12 G, 0.13 G, 0.14 G, 0.15 G, or more; or less than about 0.15 G, 0.14 G, 0.13 G, 0.12 G, 0.11 G, 0.10 G, 0.09 G, 0.08 G, 0.07 G, or less.

The motor may be assembled into the vibrational source in an orientation that may provide vibration in such ways.

In some embodiments, sensors may be added to the vibrational bruxism device, either on the vibrational device, or on the mouthpiece. The sensors may be configured to detect and monitor the parameters of the vibration, for example, frequencies and acceleration magnitudes. The sensors may also be configured to detect if the user has bitten down on the mouthpiece correctly. The sensors may be accelerometers, gyroscopes, proximity sensors, pressure sensors, humidity sensors, temperature sensors, or any combinations of them.

In some embodiments, the mouthpiece could be in contact with at least the molars or a patient's teeth nearest the TMJ. The mouthpiece may be configured to be placed in contact with a user's dentition, between and clamped down by both occlusal surfaces of the dentition. The mouthpiece can include ridges or be without ridges. The mouthpiece can cover the entire dentition, or only a part of the dentition. The shape of the mouthpiece can be customized to cover only selected teeth or implants.

Method for Treating Bruxism

According to yet another aspect of the present disclosure, a method for treating bruxism is described. The method includes providing the mouthpiece of the vibrational dental device to a user and providing instructions to the user. The instruction may include placement guidelines and dosage information. The dosage information may include duration of each treatment session, number of sessions in a day, number of days, etc. For example, the instruction may instruct a user to use the vibrational dental device for number of times per day. In some embodiments the treatment frequency can be specified to be once per day, twice per day, 3 times per day, 4 times per day, 5 times per day, 6 times per day, 7 times per day, 8 times per day, 9 times per day, or more. In some embodiments the duration of treatment can be specified to be about 1 day, 1 week, 2 weeks, 3 weeks, 1 month, 2 months, 3 months, 4 months, 5 months, 6 months, 7 months, 8 months, 9 months, 10 months, 11 months, 1 year, or more.

In some embodiments, the method may further include configuring the vibrational source providing an axial vibratory force to the mouthpiece. The axial vibratory force may be eventually applied to the dentition through the mouthpiece, which is clamped down by the teeth. The vibratory force (e.g., acceleration magnitudes, frequencies, etc.) can be adjusted by selecting preset values, or fine-tuned by users, technicians, or healthcare professionals.

According to yet another aspect of the present disclosure, a method for detecting bruxism is described. The method includes steps of identifying a patient with sleep bruxism, applying a stimulus to a portion of the dentition or alveolar ridge, applying one or more vibration sessions over a period of time, and tracking the incidence of sleep bruxism in the patient over time. In some embodiments, the method may further include modifying the LMHFV therapy in response to a patient's incidence of sleep bruxism.

### EXAMPLES

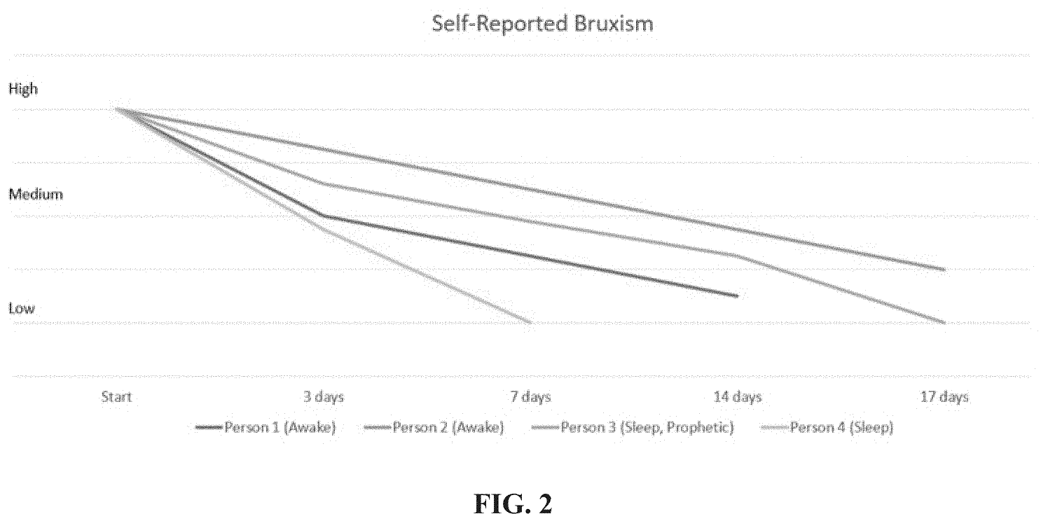

The following examples are reflected in FIG. 2 showing sleep and awake bruxism decreasing over time with use of LMHFV.

### Example 1

A patient used LMHFV at 130 Hz, initially applied for 1 session (5 minutes) in the morning and repeated in the evening daily. During an acute incident of bruxism, a 3rd session of LMHFV treatment was added at peak bruxism to aid in breaking the cycle. An immediate decrease in muscle tension and related soreness was noted after each session of LMHFV with improvement in comfort and less associated pain. Following 3 days of use twice daily, it was noted a lower incidence of awake bruxism and when occurring, the magnitude was lower. After 14 days of daily use twice daily, the incidence of awake bruxism had greatly reduced and was of lower magnitude, so use was switched to once daily as maintenance. When an acute episode occurred an additional session of LMHFV was added at the time of the incident or as soon as was practical.

### Example 2

A patient used the LMHFV device as above for 2½ weeks. He has utilized an orthotic mandibular repositioning device to increase airway space, decreasing sleep apnea. This sleep appliance prevents night-time clenching but cannot be worn during the day when clenching is noted. After the first use of the LMHFV device, there was an immediate decrease in discomfort in the masseter muscles, where the primary effect of clenching was observed. With twice a day, five-minute usage, he noted there was no more discomfort in the musculature.

### Example 3

A patient is diagnosed with sleep bruxism. Waking LMHFV is utilized by the patient daily. The patient reported no pain following treatment. The incidence of sleep bruxism decreases over time. LMHFV application is maintained to preserve the decrease in incidence of sleep bruxism.

The patient uses LMHFV at 130 Hz, initially applied for 1 session (5 minutes) in the morning and repeated in the evening daily. An immediate decrease in muscle tension and related soreness is noted after each session of LMHFV with improvement in comfort and less associated pain. Following 3 days of use twice daily, it is noted a lower incidence of sleep bruxism. After 14 days of daily use twice daily, the incidence of awake bruxism is greatly reduced and was of lower magnitude, with an attendant decrease in orofacial pain.

### Example 4

A patient was diagnosed with bruxism and was suffering from facial muscular discomfort associated with bruxism, which had developed into a chronic TMJ issue with a limited mouth opening of 22 mm. LMHFV device was utilized by the patient for 5 minutes per day for a week. The patient reported a reduction in bruxism, an improvement in sleep quality, and a relief of muscle pain around the TMJ. Mouth opening increased to 33 mm after 1 week of daily usage of LMHFV.

The foregoing descriptions have been presented for purposes of illustration. They are not exhaustive and are not limited to precise forms or embodiments disclosed. Modifications and adaptations of the embodiments will be apparent from consideration of the specification and practice of the disclosed embodiments. For example, the described implementations include hardware, but systems and methods consistent with the present disclosure can be implemented with hardware and software. In addition, while certain components have been described as being coupled to one another, such components may be integrated with one another or distributed in any suitable fashion.

Moreover, while illustrative embodiments have been described herein, the scope includes any and all embodiments having equivalent elements, modifications, omissions, combinations (e.g., of aspects across various embodiments), adaptations or alterations based on the present disclosure. The elements in the claims are to be interpreted broadly based on the language employed in the claims and not limited to examples described in the present specification or during the prosecution of the application, which examples are to be construed as nonexclusive. Further, the steps of the disclosed methods can be modified in any manner, including reordering steps or inserting or deleting steps.

It should be noted that, the relational terms herein such as “first” and “second” are used only to differentiate an entity or operation from another entity or operation, and do not require or imply any actual relationship or sequence between these entities or operations. Moreover, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items.

The features and advantages of the disclosure are apparent from the detailed specification, and thus, it is intended that the appended claims cover all systems and methods falling within the true spirit and scope of the disclosure. As used herein, the indefinite articles “a” and “an” mean “one or more.” Similarly, the use of a plural term does not necessarily denote a plurality unless it is unambiguous in the given context. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

As used herein, unless specifically stated otherwise, the terms “and/or” and “or” encompass all possible combinations, except where infeasible. For example, if it is stated that a database may include A or B, then, unless specifically stated otherwise or infeasible, the database may include A, or B, or A and B. As a second example, if it is stated that a database may include A, B, or C, then, unless specifically stated otherwise or infeasible, the database may include A, or B, or C, or A and B, or A and C, or B and C, or A and B and C.

It is appreciated that the above-described embodiments can be implemented by hardware, or software (program codes), or a combination of hardware and software. If implemented by software, it may be stored in the above-described computer-readable media. The software, when executed by the processor can perform the disclosed methods. The computing units and other functional units described in this disclosure can be implemented by hardware, or software, or a combination of hardware and software. One of ordinary skill in the art will also understand that multiple ones of the above-described modules/units may be combined as one module/unit, and each of the above-described modules/units may be further divided into a plurality of sub-modules/sub-units.

In the foregoing specification, embodiments have been described with reference to numerous specific details that can vary from implementation to implementation. Certain adaptations and modifications of the described embodiments can be made. Other embodiments can be apparent to those skilled in the art from consideration of the specification and practice of the disclosure disclosed herein. It is intended that the specification and examples be considered as exemplary only, with a true scope and spirit of the disclosure being indicated by the following claims. It is also intended that the sequence of steps shown in figures are only for illustrative purposes and are not intended to be limited to any particular sequence of steps. As such, those skilled in the art can appreciate that these steps can be performed in a different order while implementing the same method.

What is claimed is:

1. A method for treating bruxism, comprising:
identifying a patient having bruxism;
providing to the patient a vibrational dental device having a mouthpiece for contacting the dentition; and
providing instructions for using the vibrational dental device, the instruction comprising:
placing the mouthpiece over the dentition;
applying a vibratory force during a predetermined number of sessions throughout a predetermined treatment period to the patient while awake;
wherein the incidence of bruxism is less than without vibratory treatment.

2. The method of claim 1, wherein the frequency is ranging from 45 Hz to 150 Hz.

3. The method of claim 1, wherein the duration is ranging from 30 seconds to 20 minutes.

4. The method of claim 1, wherein the acceleration of the mouthpiece ranged from 0.010 G to 0.15 G.

5. The method of claim 2, wherein the frequency is about 113 Hz.

6. The method of claim 1, wherein the session time is from 30 seconds to 20 minutes.

7. The method of claim 1 where sessions are repeated daily/every other day/semi-weekly/weekly.

8. The method of claim 1, wherein the treatment period is from 1 day to 1 year.

9. The method of claim 1, wherein the bruxism treated is awake bruxism.

10. The method of claim 1, wherein the bruxism treated is sleep bruxism.

11. The method of claim 1, further comprising:
monitoring the incidence of bruxism in the patient; and
adjusting the dosage or parameters of the LMHFV based upon the results of the monitoring.
